(19) United States
(12) Patent Application Publication
(10) Pub. No.: US 2014/0377240 A1
(43) Pub. Date: Dec. 25, 2014

(54) METHODS AND COMPOSITIONS FOR EXPANDING IMMUNOSUPPRESSIVE T REGULATORY CELLS IN VITRO AND USES THEREOF

(71) Applicant: Northeastern University, Boston, MA (US)

(72) Inventors: Michail Sitkovsky, Boston, MA (US); Akio Ohta, Newton, MA (US); Akiko Ohta, Newton, MA (US)

(73) Assignee: Northeastern University, Boston, MA (US)

(21) Appl. No.: 14/372,653

(22) PCT Filed: Jan. 17, 2013
(86) PCT No.: PCT/US2013/021948

§ 371 Date: Jul. 16, 2014

Related U.S. Application Data
(60) Provisional application No. 61/587,329, filed on Jan. 17, 2012.

(51) Int. Cl. C12N 5/0783; A61K 35/14

(52) U.S. Cl. CPC C12N 5/0637; A61K 35/17; C12N 2501/2302; C12N 2500/02; C12N 2501/01
USPC 424/93.71; 435/375; 435/372.3

(57) ABSTRACT

Disclosed herein are methods and compositions for expanding T-regulatory cells (“Treg” cells), resulting in “conditioned Treg cells.” Also disclosed herein are methods and compositions useful for modulating an autoimmune reaction and for treating or ameliorating immune-related diseases, disorders and conditions using the conditioned Treg cells.

### CROSS-REFERENCE TO RELATED PATENT APPLICATIONS

This application claims the benefit of and priority to U.S. Application No. 61/587,329, filed Jan. 17, 2012, the entire contents of which is incorporated herein by reference in its entirety.

### GOVERNMENT SUPPORT

This invention was made with government support under 2R01CA111985-06-A1 awarded by the National Institutes of Health. The government has certain rights in the invention.

### BACKGROUND

Immune tolerance is central to the immune system's ability to differentiate between self and foreign proteins. Central tolerance is initially achieved during thymic selection by the deletion of self-reactive T cells. However, central tolerance is incomplete, and further immune regulation is required in the periphery. Peripheral mechanisms of T cell regulation include the induction of anergy, activation induced cell death, and regulatory T cells (also know as T-regulatory cells or “Treg” cells).

Within the CD4⁺ T lymphocyte cell population, several categories of regulatory T cells have been described. In general, these subpopulations are classified according their site of development and/or the cytokines they produce. One subset of regulatory T cells develops in the thymus (natural regulatory T cells) while a different subset differentiates from CD4⁺ CD25⁻ precursors after leaving the thymus and encountering specific antigen in the periphery (inducible regulatory T cells). Among inducible regulatory T cell subsets, Tr1 cells secrete IL-10, while Th3 cells secrete TGF-β, although both cell types have been shown to produce both IL-10 and TGF-β to some extent. More recently, investigators have shown that the expression of forkhead box protein P3 (“FoxP3”) transcription factor is an important marker in the classification of regulatory T cells.

### SUMMARY

Disclosed herein are novel compositions and methods useful to differentiate and expand, ex vivo, immunosuppressive T regulatory cells (“Treg” cells). Also disclosed herein are methods and compositions useful to treat, ameliorate or modulate immune related diseases and conditions, including autoimmune disease such as diabetes, and diseases, conditions and complications resulting from transplantation, such as but not limited to, bone marrow, organ and tissue transplantation, among others.

In some aspects, a method for expanding T-regulatory cells is provided. In some embodiments, the method includes culturing T-regulatory cells for at least 3 days under the following conditions: 0.5-5% oxygen, 5-100 U/ml of IL-2, and in the presence of anti-CD3 and anti-CD28 antibodies. In some embodiments, the T-regulatory cells to be expanded are CD4 positive and CD25 positive. In some embodiments, at least 90% of the CD4 positive and CD25 positive T-regulatory cells are FoxP3 positive. In some embodiments, at least 95% of the CD4 positive and CD25 positive T-regulatory cells are FoxP3 positive. In some embodiments, the T-regulatory cells comprise human cells. In some embodiments, the T-regulatory cells are cultured under 1% oxygen.

In some embodiments, T-regulatory cells are isolated (e.g., from the culture medium) after culturing. In some embodiments, T-regulatory cells are isolated after at least 3 days of culture. Additionally or alternatively, in some embodiments, T-regulatory cells expressing increased CTLA-4 and/or increased IL-10 levels as compared to control T-regulatory cells are isolated (e.g., from the culture medium, and/or from cells not expressing increased CTLA-4 and/or IL-10 levels).

In some embodiments, the T-regulatory cells are contacted with an agent that increases intracellular cyclic AMP (cAMP) levels.

In some aspects, a method for expanding T-regulatory cells is provided. In some embodiments, the method includes (a) culturing T-regulatory cells under normoxic conditions; culturing the T-regulatory cells of step (a) for at least 3 days under the following conditions: 0.5-5% oxygen, 5-100 U/ml of IL-2, and in the presence of anti-CD2 and anti-CD28 antibodies. In some embodiments, the T-regulatory cells to be expanded are CD4 positive and CD25 positive. In some embodiments, at least 90% of the CD4 positive and CD25 positive cells are FoxP3 positive. In some embodiments, the T-regulatory cells comprise human cells. In some embodiments, the T-regulatory cells are cultured under 1% oxygen. In some embodiments, the T-regulatory cells are contacted with an agent that increases intracellular cyclic AMP (cAMP) levels.

In some aspects, a method for expanding T-regulatory cells is provided. In some embodiments, the method includes culturing T-regulatory cells for at least 3 days in the presence of an agent that increases intracellular cyclic AMP (cAMP) levels. In some embodiments, the agent that increases intracellular cAMP levels includes one or more G protein-coupled receptor ligands. In some embodiments, the G protein-coupled receptor ligand includes one or more of: ligands of the A2A and A2B receptor (adenosine), ligands of the β-adrenergic receptor ligands (adrenaline), ligands of D1 and D5 receptors (dopamine), ligands of H2 receptor (histamine), ligands of DP, IP, EP2 and EP4 receptors (prostaglandins), ligands of 5-HT4, 5-HT6, 5-HT7 receptors (serotonin), ligands of PACT, VPAC1, VPAC2 and ligands of glucagon receptors (VIP, PACAP, glucagon). In some embodiments, the compound that increases intracellular cAMP levels includes one or more of phosphodiesterase inhibitors, ibudilast, cholera toxin, forskolin, caffeine, theophylline, bucladesine, dibutyryl cAMP, db cAMP, pertussis toxin, milrinone, inamrinone, sildenafil, tadalafil, and activators of Gs protein. In some embodiments, the T-regulatory cells comprise human cells.

In some aspects, a method for modulating an autoimmune reaction in a subject in need thereof is provided. In some embodiments, the method includes administering T-regulatory cells expanded by one or more of the methods described above. In some embodiments, the T-regulatory cell is obtained from the subject prior to expanding. In some embodiments, the subject is suffering from an autoimmune disease, such as, but not limited to Addison's disease, Celiac disease, dermatomyositis, Graves disease, Hashimoto's thyroiditis, multiple sclerosis, myasthenia gravis, pernicious anemia, reactive arthritis, rheumatoid arthritis, Sjogren syndrome, systemic lupus erythematosus, type I diabetes, graft versus host disease after solid organ transplant or bone marrow transplant.

### BRIEF DESCRIPTION OF THE DRAWINGS

FIG. 1 is a chart showing T-regulatory (“Treg”) cell proliferation under hypoxic cell culture conditions in response to IL-2. The data represents cell numbers after three days of hypoxic culture.

FIG. 2 is a chart showing the up-regulation of CTLA-4 expression in conditioned Treg cells cultured under hypoxic conditions for 3 days. The numbers represent the percentage of FoxP3⁺ CTLA-4⁺ and FoxP3⁺ CTLA-4⁻ cells present under each condition. Hypoxia increased Treg cell expression of CTLA-4 irrespective of IL-2 concentration, as shown by the proportion of CTLA-4⁺ cells present and by mean fluorescence intensity (MFI).

FIG. 3 is a chart showing increased IL-10 production by conditioned Treg cells cultured under hypoxic conditions. IL-10 levels were determined in the culture supernatant following 3 days of hypoxic culture.

FIG. 4 is a chart showing that hypoxia promotes the immunoregulatory activity of conditioned Treg cells.

### DETAILED DESCRIPTION

Given the important role CD4⁺ (CD4 positive) CD25⁺ (CD25 positive) T-regulatory (“Treg”) cells play in immune tolerance, there is a need to develop methods for generating, selecting, and expanding human antigen-specific CD4⁺CD25⁺ Treg cells. Such cells may be isolated from the peripheral blood of a subject, for example, and used in the treatment and/or prevention of autoimmune disorders, allergies, inflammatory conditions and for the prevention of graft rejection in a recipient following solid organ, tissue, bone marrow, or stem cell transplantation.

Accordingly, disclosed herein are methods and compositions for expanding Treg cells, resulting in “conditioned Treg cells.” Also disclosed herein are methods and compositions useful for treating autoimmune diseases and disorders using conditioned Treg cells expanded by the methods disclosed herein.

The technology is described herein using several definitions, as set forth throughout the specification.

As used herein, the singular forms “a,” “an,” and “the” include plural referents unless the context clearly dictates otherwise. Thus, for example, reference to “a pharmaceutical carrier” includes mixtures of two or more such carriers, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “aberrant immune response” refers to the failure of a subject's immune system to distinguish self from non-self or the failure to respond to foreign antigens. The term also embraces hyperimmune responses to foreign antigens as in the case of allergic disorders. Thus, the response is present in both autoimmune disorders and allergic disorders. Aberrant immune responses include, but are not limited to, tissue injury and inflammation caused by the production of antibodies to an organism's own tissue, impaired production of cytokines and tissue damage caused by cytotoxic or non-cytotoxic mechanisms of action. In some embodiments, aberrant immune responses are inappropriately regulated immune responses that lead to patient symptoms. Typically, autoimmune responses occur when the immune system of a subject recognizes self-antigens as foreign, leading to the production of self-reactive effector immune cells. Self-reactive effector immune cells include cells from a variety of lineages, including, but not limited to, cytotoxic T cells, helper T cells, and B cells. While the precise mechanisms differ, the presence of autoreactive effector immune cells in a patient suffering from an autoimmune disorder may lead to the destruction of tissues and cells of the patient, resulting in pathologic symptoms. Similarly, the presence of cells that undergo a hypersensitive reaction to foreign antigens to which normal individuals respond in a more restrain manner is indicative of hypersensivity (allergy). Examples include, but are not limited to, food allergies, hay fever, and allergic asthma. Numerous assays for determining the presence of such cells in a patient, and therefore the presence of an autoimmune disorder, such as an antigen specific autoimmune disorder in a patient, or an allergic disorder, are known to those of skill in the art and readily employed in the subject methods.

As used herein, the term “antibody” refers to polyclonal and monoclonal antibodies, chimeric antibodies, haptens and antibody fragments, and molecules which are antibody equivalents in that they specifically bind to an epitope on the antigen of interest (e.g. counter receptors for the TCR/CD3 complex and ICAM-1). The term “antibody” includes polyclonal and monoclonal antibodies of any isotype (IgA, IgG, IgE, IgD, IgM), or an antigen-binding portion thereof, including, but not limited to, F(ab) and Fv fragments such as sc Fv, single chain antibodies, chimeric antibodies, humanized antibodies, and a Fab expression library. When used to stimulate a T cell, antibodies can also be immobilized for instance on a solid phase surface, such as a particle, or linked to the surface of a culture well or plate.

As used herein, the term “antigen” refers to any molecule capable of generating an immune response. By way of example, in the context of autoimmune disorders, the antigen is a self-antigen.

As used herein, the term “cell” refers to a single cell as well as a plurality or population of cells.

As used herein the term “control cell” refers to a cell that is not subjected to or contacted with a test agent or test condition and which serves as a reference cell to determine or evaluate differences in another cell (e.g., a test cell) which has been subject to the test agent or test condition. Typically, a control cell is the same cell type as the test cell (e.g., a Treg cell isolated from the same source using the same or similar methods). By way of example, a control cell is grown or treated via “standard” conditions or conditions typically used for cell culture, while the test cell is subject to one or more variables (e.g., hypoxic culture conditions, the presence of one or more pharmacological agents that increase intracellular cAMP levels, etc.). In some embodiments, the difference between a test cell and a control cell includes, without limitation, differences in the levels of cell surface or intracellular molecules (e.g., IL-10 and/or CTLA-4, intracellular cAMP levels, etc.) or cell activity (e.g., immunosuppressive activity).

As used herein, “immune response” refers to a patient response to foreign or self antigens. The term includes cell mediated, humoral, or inflammatory responses.

As used herein, “patient” and “subject” are used interchangeably, and refer to a mammal, for example a human. In some cases, the methods and compositions disclosed herein find use in experimental animals, in veterinary application, and in the development of animal models for disease, including, but not limited to, dogs, cats, pigs, horses, cattle, chimpanzees, monkeys, rodents including mice, rats, and hamsters, and primates.

As used herein, “proliferation” or “expansion” refers to the ability of a cell or population of cells to increase in number.

As used herein, “standard culture conditions” refers to those conditions, known in the art, which are typically used to culture a given cell type. By way of example but not by way of limitation, “standard culture conditions” for Treg cells include the following. Media: RPMI1640 (Invitrogen) supplemented with 10% fetal calf serum (Hyclone) or AIM-V serum-free medium (Invitrogen); culturing condition: 37° C., 5% CO₂ using, e.g., a NAPCO7000 incubator capable of controlling oxygen concentration; humidity: >95%, cell density: typically about 5×10⁵ cells/ml, media change after about 3 days.

As used herein, a composition containing a “purified cell population” or “purified cell composition” means that at least 30%, 50%, 60%, 70%, 80%, 90%, 95%, 98%, or 99% of the cells in the composition are of the identified type.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances. In one aspect, at least 30%, 50%, 60%, 70%, 80%, 90%, 95%, 98%, or 99% of the second substance is removed from the first substance.

As used herein, the terms “regulatory T cell,” “T-regulatory cell” and “Treg cell” are used interchangeably, and refer to T cells that express CD4⁺CD25⁺ phenotype. In some embodiments, the Treg cells also express the FoxP3 transcription factor as measured by methods known in the art, e.g., flow cytometry, Western blot, FoxP3 mRNA transcript detected in vitro or in vivo, etc. Treg cells may be obtained from a variety of mammalian sources, including, but not limited to mammals typically used in experimental settings, such as rodents (e.g., mice, rats), rabbits, goats, ferrets, monkeys and apes, common domestic animals such as cattle, horses, sheep, hogs, dogs, cats, and other mammals, such as those kept in zoos or as pets, etc. I some embodiments, Treg cells are human cells.

As used herein the term “conditioned” with reference to Treg cells includes (a) isolated Treg cells that have been expanded and cultured, in vitro, under hypoxic conditions; (b) isolated Treg cells that have been expanded and cultured in vitro, and contacted, in vitro, with one or more agents that increase intracellular cyclic AMP (cAMP) levels; (c) isolated Treg cells that have been expanded and cultured, in vitro, under hypoxic conditions and that have been contacted, in vitro, with one or more agents that increase intracellular cAMP levels. In addition, conditioned Treg cells express increased levels of CTLA-4 as compared to control Treg cells. In some embodiments, conditioned Treg cells express increased levels of IL-10 as compared to control Treg cells. In some embodiments, conditioned Treg cells are provided as a therapeutic agents or therapeutic composition and are administered to a subject suffering from an immune disease or disorder, and/or exhibiting an aberrant immune response.

The terms “suppression,” “inhibition” and “prevention” are used herein in accordance with accepted definitions in the context of an immune response. For example, “suppression” results when an ongoing immune response is blocked or significantly reduced as compared with the level of immune response that results absent treatment, e.g., by the Treg cells disclosed herein. “Inhibition” refers to blocking the occurrence of an immune response or significantly reducing such response as compared with the level of immune response that results absent treatment, e.g., by the Treg cells disclosed herein. When administered prophylactically, such blockage may be complete so that no targeted immune response occurs, typically referred to as a “prevention” with regard to completely blocking the immune response before onset; or in the present disclosure, the treatment may advantageously reduce the effect as compared to the normal untreated state, typically referred to as suppression or inhibition.

As used herein, “therapeutically effective amount” refers to an amount, e.g., of a therapeutic composition, that is sufficient to treat or ameliorate, begin to palliate, stabilize, reverse or slow progression of a disease, or otherwise reduce pathological consequences of the disease or in some manner reduce the symptoms associated with a disease or disorder. In any case, an effective amount may be given in single or divided doses. The term “therapeutically effective,” when used with reference to a method, means that the method is sufficiently effective to treat or ameliorate, begin to palliate, stabilize, reverse or slow progression of a disease, or otherwise reduce pathological consequences of the disease or in some manner reduce the symptoms associated with a disease or disorder.

As used herein, the term “treatment” refers to at least an amelioration of the symptoms associated with the aberrant immune response in the patient is achieved, where amelioration is used in a broad sense to refer to at least a reduction in the magnitude of a parameter, e.g. symptom, associated with the condition being treated. As such, “treatment” also includes situations where the disease, disorder, or pathological condition, or at least symptoms associated therewith, are completely inhibited, e.g. prevented from happening, or stopped, e.g. terminated, such that the patient no longer suffers from the condition, or at least the symptoms that characterize the condition.

### I. CONDITIONED TREG CELLS

In one aspect, the present disclosure is directed to a method of expanding Treg cells, to provide conditioned Treg cells. In some embodiments, a subject's own T cells are collected, enriched, and subjected to expansion protocols according to the methods disclosed herein. In some embodiments, the “conditioned” Treg cells are then administered to the subject, e.g., to treat an immune, autoimmune or allergic disorder, or to treat or ameliorate an aberrant immune response.

A. Collection and Enrichment of Treg Cells

Treg cells may be obtained from a variety of mammalian sources, including, but not limited to mammals typically used in experimental settings, such as rodents (e.g., mice, rats), rabbits, goats, ferrets, monkeys and apes, common domestic animals such as cattle, horses, sheep, hogs, dogs, cats, and other mammals, such as those kept in zoos or as pets, etc. In some embodiments, Treg cells are isolated from a sample of a subject's peripheral blood. In some embodiments, the subject is a human and the Treg cells are human cells. Methods for collecting blood samples and isolating cells are well known in the art.

In some embodiments, Treg cells are substantially separated from the other cells in the blood sample to form a purified Treg cell population. Methods for isolating and purifying Treg cells are well known in the art. By way of example, but not by way of limitation, methods may be based on using monoclonal antibodies against cell surface proteins which are predominantly expressed on Treg cells. For example, using fluorochrome-conjugated antibodies, Treg cells can be labeled and isolated, e.g., by magnetic cell sorting, flow cytometry, etc., (see e.g., Kawano Y, et al. 2011. Blood 118:5021-5030.)

In some embodiments, Treg cells are isolated and enriched for CD4 positive, CD25 positive cells. By way of example, but not by way of limitation, in some embodiments, human peripheral blood mononuclear cells are separated from peripheral blood by density centrifugation using Ficoll. In some embodiments, peripheral blood mononuclear cells are labeled with anti-CD4, anti-CD25 and anti-CD127 antibodies and CD4 positive, CD25 med-hi, CD127 low cells are isolated as Treg by, e.g., FACS Aria II Cell Sorter. In some embodiments, cells are further enriched for FoxP3. In some embodiments, about 85%, 86%, 87%, 88%, 89%, 90%, 91%, 92%, 93%, 94%, 95%, 96%, 97%, 98%, 99% of the isolated and enriched Treg cells are CD4 positive and CD25 positive. In some embodiments, about 93% or greater, e.g., about 94%, 95%, 96%, 97%, 98%, 99% of the isolated and enriched Treg cells are CD4 positive and CD25 positive. In some embodiments, about 85%, 86%, 87%, 88%, 89%, 90%, 91%, 92%, 93%, 94%, 95%, 96%, 97%, 98%, 99% of the isolated and enriched CD4 positive, CD25 positive cells are FoxP3 positive. In some embodiments, about 95% of the isolated and enriched CD4 positive, CD25 positive cells are FoxP3 positive.

In some embodiments, the Treg cells may be selected against dead cells by employing dyes associated with dead cells (e.g., propidium iodide, ethidium monoazaide). Any technique may be employed which is not unduly detrimental to the viability of the selected cells.

In some embodiments, cell sorting is used. In some embodiments, the Treg cells may be collected in any appropriate medium that maintains the viability of the cells, usually having a cushion of serum at the bottom of the collection tube. Various media are commercially available and may be used according to the nature of the cells, including Dulbecco's Modified Eagle Medium (“dMEM”), Hank's Basic salt Solution (“HBSS”), Dulbecco's phosphate buffered saline (“dPBS”), RPMI, Iscove's medium, etc., frequently supplemented with fetal calf serum.

In some embodiments, at least 75%, 85%, 90%, 95%, or 98% of the cells of the resulting composition are Treg cells.

B. Expansion of Treg Cells to Yield Conditioned Treg Cells

The culture conditions disclosed herein used to expand a population of Treg cells, yield “conditioned Treg cells” which are useful as therapeutic agents. The culture conditions include one or more of the following: (a) culturing the cells, in vitro, under hypoxic conditions; and (b) exposing the cells, in vitro, to an agent which increases intracellular cyclic AMP (cAMP) level. Such conditioned Treg cells exhibit increased expression levels of CTLA-4 and/or IL-10 as compared to control Treg cells (e.g., Treg cells that were not expanded according to the methods disclosed herein).

1. Hypoxic Culture Conditions

In some embodiments, Treg cells (e.g., CD4, CD25 and FoxP3 positive cells) are cultured under standard temperature and humidity conditions, in standard growth medium, under hypoxic conditions. Such culturing results in conditioned Treg cells.

By way of example, but not by way of limitation, culture conditions for generating conditioned Treg cells include the following: hypoxic conditions; media: RPMI1640 (Invitrogen) supplemented with 10% fetal calf serum (Hyclone) or AIM-V serum-free media (Invitrogen); culturing condition: 37° C., 5% CO₂ using e.g., a NAPCO7000 incubator capable of controlling oxygen concentration to generate hypoxic conditions; humidity: >95%, cell density: typically at 5×105 cells/ml, with media change after 3 days.

As used herein, hypoxic conditions refer to an atmosphere for cell culture in which there is less than about 15% oxygen, 12% oxygen, 10% oxygen, 9% oxygen, 8% oxygen, 7% oxygen, 6% oxygen, 5% oxygen, 4% oxygen, 3% oxygen, 2% oxygen, 1% oxygen, 0.5% oxygen or substantially devoid of oxygen. In some embodiments, hypoxic conditions include oxygen at about 0-15%, about 0-10%, about 0-5%, about 0-3%, about 0-1%. In some embodiments, hypoxic conditions include oxygen at about 0.5-15%, about 0.5-10%, about 0.5-5%, about 0.5-3%, about 0.5-1%. In some embodiments, hypoxic conditions include oxygen at about 1-15%, about 1-10%, about 1-5%, about 1-3%, about 1-2%. In some embodiments, hypoxic conditions include oxygen at about 2-15%, about 2-10%, about 2-5%, about 2-3%. In some embodiments, hypoxic conditions include oxygen at about 3-15%, about 3-10%, about 3-5%, about 3-4%. In some embodiments, hypoxic conditions include oxygen at about 4-15%, about 4-10%, about 4-5%. In some embodiments, hypoxic conditions include oxygen at about 5-15%, about 5-10%. In some embodiments, hypoxic conditions include 1% oxygen. To avoid confusion, standard, non-hypoxic (“normoxic”) incubation conditions for cell culture typically includes 95% air (21% oxygen) and about 5% CO₂.

In some embodiments, a population of Treg cells (e.g., CD4, CD25 and FoxP3 positive cells) is cultured and expanded under hypoxic conditions in the presence of a stimulating agent, such as a T-cell receptor (“TCR”)/CD3 activator. In some embodiments, the TCR/CD3 activator includes an antibody, such as an anti-CD3 antibody. In some embodiments, the anti-CD3 antibody comprises a polyclonal antibody. In some embodiments, the anti-CD3 antibody comprises a monoclonal antibody. A number of anti-CD3 monoclonal antibodies are commercially available, e.g., OKT3 and G19-4 monoclonal antibodies prepared from hybridoma cells obtained from the American Type Culture Collection.

Additionally or alternatively, in some embodiments, a population of Treg cells is cultured and expanded under hypoxic conditions in the presence of anti-CD28 antibodies. In some embodiments, the anti-CD28 antibody comprises a polyclonal antibody. In some embodiments, the anti-CD28 antibody comprises a monoclonal antibody.

In some embodiments, the stimulating agents (e.g., anti-CD3 and anti-CD28 antibodies) may be in soluble form or immobilized on a solid support, such as a bead or tissue culture dish. Antibodies may be added at about 0.005-2 μg/ml. For example, antibodies may be added at 0.1 μg/ml (anti-CD3 antibody) and 1 μg/ml (anti-CD28 antibody) as soluble form. When the antibodies are to be immobilized on a tissue culture plasticware (e.g., a tissue culture plate or dish), both anti-CD3 and CD28 antibodies may be added at 1 μg/ml. Microbeads conjugated with anti-CD3 and anti-CD28 antibodies are commercially available and may be used according to manufacturer's instruction. For example, in some embodiments, the two stimulating agents are coupled to the same solid phase surface, such as a bead, or the bottom of a culture dish or well. The solid phase surface can be plastic, glass, or any other suitable material. In some embodiments, paramagnetic beads are used, and are typically in the 1-20 micron range.

Additionally or alternatively, in some embodiments, the stimulating agent includes other antibodies which activate expansion of Treg cells, and/or includes antigen presenting cells which activate Treg cells.

Additionally or alternatively, in some embodiments, a population of Treg cells is cultured and expanded under hypoxic conditions in the presence of one or more agents, including IL-2 (5-100 U/ml, e.g., 20 U/ml or 100 U/ml), IL-7 (1-100 ng/ml, e.g., 10 ng/ml), IL-10 (1-100 ng/ml, e.g., 10 ng/ml), TGF-beta (1-100 ng/ml, e.g., 5 ng/ml), glucocorticoid-induced TNF-α receptor-related protein ligand (GITR-L) (1-100 ng/ml, e.g., 20 ng/ml). In some embodiments, one or more of the agents listed above, e.g., IL-2, is present during expansion/culture for the entire culture period. In some embodiments, IL-2 is present during expansion/culture at about 5, 10, 20, 30, 40, 50, 60 70, 70 90 or 100 U/ml for the entire culture period.

In some embodiments, Treg cells are cultured under hypoxic conditions, in the presence of anti-CD3 and anti-CD28 antibodies and 5-100 U/ml of IL-2 for at least 3 days. In some embodiments, Treg cells are incubated for about 4, 5, 6, 7, 8, 9, 10, 11, 12, 13, 14, 15, 16, 17, 18, 19, 20 or 21 days. In some embodiments, cells are incubated for about 3-7 days.

In some embodiments, a population of Treg cells (e.g., CD4, CD25 and FoxP3 positive cells) is first incubated (cultured) under normoxic conditions, and is then incubated (cultured) under hypoxic conditions. In some embodiments, the hypoxic culture conditions include culturing the cells in the presence of IL-2, anti-CD3 antibodies and anti-CD28 antibodies. In some embodiments, normoxic culturing is for less than 3 days. For example, in some embodiments, cells are cultured under normoxic conditions for about 1 day, about 2 days, or about 3 days. In some embodiments, cells are cultured under normoxic conditions for less than about 5 days, e.g., about 4 days. In some embodiments, cells are cultured under normoxic conditions for 6, 7, 8, 9, 10, 11, 12, 13 or 14 days.

2. Cyclic AMP Elevation

In some embodiments, Treg cells (e.g., CD4, CD25 and FoxP3 positive cells) are cultured in the presence of one or more agents that increase intracellular cAMP levels. Such culturing results in conditioned Treg cells, which express increased levels of CTLA-4 as compared to control Treg cells.

Numerous agents that increase intracellular cAMP levels are known in the art, and exemplary non-limiting compounds include G protein-coupled receptor ligands such as ligands of the A2A and A2B receptor (adenosine), ligands of the β-adrenergic receptor ligands (adrenaline), ligands of D1 and D5 receptors (dopamine), ligands of H2 receptor (histamine), ligands of DP, IP, EP2 and EP4 receptors (prostaglandins), ligands of 5-HT4, 5-HT6, 5-HT7 receptors (serotonin), ligands of PACT, VPAC1, VPAC2 and glucagon receptors (VIP, PACAP, glucagon). Additional exemplary agents include, without limitation phosphodiesterase inhibitors (including ibudilast), cholera toxin, forskolin, caffeine, theophylline, bucladesine (dibutyryl cAMP, db cAMP), pertussis toxin, inhibitors of cyclic AMP dependent phosphodiesterase (PDE), and activators of Gs protein Inhibitors of cyclic AMP dependent phosphodiesterase (PDE) include but are not limited to PDE3 inhibitors (e.g., milrinone, inamrinone (formerly amrinone), cilostazol), PDE4 inhibitors (e.g. Ibudilast, roflumilast) and PDE5 inhibitors (e.g., sildenafil, tadalafil).

In some embodiments, intracellular cAMP levels are increased by 5-fold or more as compared to control Treg cells not contacted with the agent (e.g., not contacted with ligands of adenosine receptor). In some embodiments, intracellular cAMP levels are increased about 2-fold, 3-fold, 4-fold, 5-fold, 6-fold, 7-fold, 8-fold, 9-fold, 10-fold, 15-fold, 20-fold, 25-fold or 30-fold or more over control cAMP levels.

In some embodiments, the population of Treg cells is cultured under standard culture conditions (e.g., standard temperature, humidity, medium and oxygen) when exposed to the cAMP inducer. In some embodiments, the population of Treg cells is cultured under hypoxic conditions (e.g., standard temperature, humidity and medium, but oxygen at 0.5-5%) when exposed to the cAMP inducer.

In some embodiments, the population of Treg cells is first cultured under normoxic conditions, and then is cultured under hypoxic conditions. In some embodiments, the Treg cells are exposed to the cAMP inducer during the normoxic culture, during the hypoxic culture, or both.

In some embodiments, Treg cells are exposed to about 0.1 nM to about 0.1 mM of cAMP inducer. In some embodiments, cells are exposed to the cAMP inducer for 1, 2, 3, 4, 5, 6, 7, 8 9, 10, 11, 12, 13, 14 or more days during culture. In some embodiments, cells are exposed to the cAMP inducer continuously throughout the culture period. In some embodiments, cells are exposed to cAMP inducer periodically throughout the culture period (e.g., every other day, every second or third day, for only a few hours each day, etc.).

C. Conditioned Treg Cells and Compositions Comprising the Cells

In some embodiments, the Treg cells cultured as described above are expanded at least 2-fold, at least 3-fold, 4, 5, 6, 7, 8, 9, 10, 50, 100, 200, 300, 500, or at least 800-fold. In some embodiments, the expanded conditioned Treg cells are then harvested or isolated.

In some embodiments, compositions comprising the conditioned Treg cells contain a clinically relevant number or population of Treg cells. In some embodiments, compositions include about 10⁵ cells, about 10⁶ cells, about 10⁷ cells, about 10⁸ cells, about 10⁹ cells, about 10¹⁰ cells or more. In some embodiments, the number of cells present in the composition will depend upon the ultimate use for which the composition is intended, e.g., the disease or state or condition, patient condition (e.g., size, weight, health, etc.), and other health-related parameters that a skilled artisan would readily understand. In addition, in some embodiments, the clinically relevant number of cells can be apportioned into multiple infusions that cumulatively equal or exceed the desired administration, e.g., 10⁹ or 10¹⁰ cells.

In some embodiments, compositions including the cells also include a pharmaceutical carrier, antibiotics or other active agents that would facilitate patient treatment.

In some embodiments, the conditioned Treg cell population may be used immediately. In some embodiments, cells can be frozen at liquid nitrogen temperatures and stored for long periods of time, being thawed and capable of being reused. The cells may be stored, for example, in DMSO and/or FCS, in combination with medium, glucose, etc. Once thawed, the cells may be expanded by use of growth factors, antigen-stimulation, cytokines dendritic cells, etc.

### II. EXEMPLARY USES OF CONDITIONED TREG CELLS

In some embodiments, the compositions of the present disclosure comprising conditioned Treg cells are useful for suppression of immune function in a patient. For example, as describe above, autologous cells may be isolated, expanded and cultured in vitro as described herein, and subsequently administered or re-introduced to the patient. In some embodiments, such treatment is useful for example, to down-regulate harmful T cell responses to self and foreign antigens, and/or to induce long term tolerance.

In some embodiments, a therapeutically effective amount of a composition comprising conditioned Treg cells as disclosed herein can be administered to the subject with a pharmaceutically acceptable carrier. Administration routes may include any suitable means, including, but not limited to intravascularly (intravenously or intra-arterially). In some embodiments, a preferred administration route is by IV infusion. In some embodiments, the particular mode of administration selected will depend upon the particular treatment, disease state or condition of the patient, the nature or administration route of other drugs or therapeutics administered to the subject, etc.

In some embodiments, about 10⁹-10¹¹ cells can be administered in a volume of a 50 ml to 1 liter, 50 ml to 250 ml, 50 ml to 150, and typically 100 ml. In some embodiments, the volume will depend upon the disorder treated, the route of administration, the patient's condition, disease state, etc. The cells can be administered in a single dose or in several doses over selected time intervals, e.g., to titrate the dose.

In one aspect, the compositions and methods disclosed herein are directed to modulating an aberrant immune response in a subject, such as an autoimmune disorder or an allergy, by administering the Treg compositions disclosed herein. In some embodiments, the subject is suffering from an autoimmune disorder or an allergic response, and the Treg compositions are used to treat the autoimmune disorder or allergic disorder. In some embodiments, the subject is an animal model of an autoimmune disorder or allergic disorder. Some embodiments, the subject is a human afflicted with an autoimmune disorder or allergic disorder.

The conditioned Treg compositions disclosed herein are used to treat, alleviate or ameliorate the symptoms of or suppress a wide variety of autoimmune disorders. In some embodiments, the autoimmune disorders including but not limited to Addison's disease, Alopecia universalis, ankylosing spondylitisis, antiphospholipid antibody syndrome, aplastic anemia, asthma, autoimmune hepatitis autoimmune infertility, autoimmune thyroiditis, autoimmune neutropenia, Behcet's disease, bullous pemphigoid, Chagas' disease, cirrhosis, Cocliac disease, colitis, Crohn's disease, Chronic fatigue syndrome, chronic active hepatitis, dense deposit disease, discoid lupus, degenerative heart disease, dermatitis, insulin-dependent diabetes mellitus, dysautonomia, endometriosis, glomerulonephritis, Goodpasture's disease, Graves' disease, graft versus host disease (GVHD), graft rejection in a recipient following solid organ (e.g., heart, liver, kidney, lung), tissue, bone marrow, or stem cell transplantation, Graves' disease, Guillain-Barre syndrome, Hashimoto's disease, hemolytic anemia, Hidradenitis suppurativa, idiopathic thrombocytopenia purpura, inflammatory bowel disease (“IBD”), insulin dependent diabetes mellitus, interstitial cystitis, mixed connective tissue disease, multiple sclerosis (“MS”), myasthenia gravis, neuromyotonia, opsoclonus myoclonus syndrome, optic neuritis, Ord's thyroiditis, pemphigus vulgaris, pernicious anemia, polyarthritis, polymyositis, primary biliary cirrhosis, psoriasis, Reiter's syndrome, rheumatoid arthritis (“RA”), sarcoidosis, scleroderma, Sjogren's syndrome, systemic lupus erythematosus, Takayasu's arteritis, temporal arteritis, thrombocytopenia purpura, ulcerative colitis, vitiligo, vulvodynia, warm autoimmune hemolytic anemia, or Wegener's granulomatosis.

Additionally or alternatively, in some embodiments, the conditioned Treg compositions disclosed are used to treat, alleviate or ameliorate the symptoms of or suppress a wide variety of immune related diseases or conditions. In some embodiments, the immune related disease or condition includes, without limitation allergic conjunctivitis, allergic rhinitis, allergic contact dermatitis, anaphylactoid purpura, asthma, erythema elevatum diutinum, erythema marginatum, erythema multiforme, allergic granulomatosis, granuloma annulare, granlocytopenia, hypersensitivity pneumonitis, keratitis, neplirotic syndrome, overlap syndrome, pigeon breeder's disease, pollinosis, idiopathic polyneuritis, urticaria, uveitis, juvenile dermatomyositis, acute disseminated encephalomyelitis (adem), Addison's disease, agammaglobulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease/Balo concentric sclerosis, Behçet's disease, Berger's disease, Bickerstaffs encephalitis, Blau syndrome, bullous pemphigoid, cancer, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, crest syndrome, Crohn's disease, Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressiva, fibrosing alveolitis (idiopathic pulmonary fibrosis), gastritis, gastrointestinal pemphigoid, glomerulonephritis, Goodpasture's syndrome, Graves' disease, Guillain-Barré syndrome (GBS), Hashimoto's encephalopathy, Hashimoto's thyroiditis, Henoch-Schonlein purpura, herpes gestationis (gestational pemphigoid), hidradenitis suppurativa, Hughes-Stovin syndrome, hypogammaglobulinemia, idiopathic inflammatory demyelinating diseases, idiopathic pulmonary fibrosis, idiopathic thrombocytopenic purpura (autoimmune thrombocytopenic purpura), IgA nephropathy, inclusion body myositis, chronic inflammatory demyelinating polyneuropathy, interstitial cystitis, juvenile idiopathic arthritis (juvenile rheumatoid arthritis), Kawasaki's disease, Lambert-Eaton myasthenic syndrome, leukocytoclastic vasculitis, lichen planus, lichen sclerosus, linear IgA disease (lad), Lou Gehrig's disease (Amyotrophic lateral sclerosis), lupoid hepatitis (autoimmune hepatitis), lupus erythematosus, Majeed syndrome, Méniére's disease, microscopic polyangiitis, Miller-Fisher syndrome (Guillain-Barre Syndrome), mixed connective tissue disease, morphea, Mucha-Habermann disease (Pityriasis lichenoides et varioliformis acuta), multiple sclerosis, myasthenia gravis, myositis, narcolepsy, neuromyelitis optica (devic's disease), neuromyotonia, occular cicatricial pemphigoid, opsoclonus myoclonus syndrome, Ord's thyroiditis, palindromic rheumatism, pandas (pediatric autoimmune neuropsychiatric disorders associated with streptococcus), paraneoplastic cerebellar degeneration, paroxysmal nocturnal hemoglobinuria (pnh), Parry Romberg syndrome, Parsonage-Turner syndrome, pars planitis, pemphigus vulgaris, pernicious anaemia, perivenous encephalomyelitis, poems syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome, Schnitzler syndrome, scleritis, scleroderma, serum sickness, Sjögren's syndrome, spondyloarthropathy, Still's disease (Juvenile Rheumatoid Arthritis), stiff person syndrome, subacute bacterial endocarditis (sbe), Susac's syndrome, Sweet's syndrome, Sydenham chorea see PANDAS, sympathetic ophthalmia, systemic lupus erythematosis, Takayasu's arteritis, temporal arteritis (giant cell arteritis), thrombocytopenia, Tolosa-Hunt syndrome, transverse myelitis, ulcerative colitis, undifferentiated connective tissue disease, undifferentiated spondyloarthropathy, urticarial vasculitis, vasculitis, vitiligo, wegener's granulomatosis, graft versus host disease (GVHD).

In some embodiments, the conditioned Treg cell compositions disclosed herein are used to treat, alleviate or ameliorate the symptoms of or suppress a wide variety of allergic disorders including, but not limited to, allergic conjunctivitis, allergic rhinitis, allergic contact dermatitis, alopecia universalis, anaphylactoid purpura, asthma, atopic dermatitis, dermatitis herpetiformis, erythema elevatum diutinum, erythema marginatum, erythema multiforme; erythema nodosum, allergic granulomatosis, granuloma annulare, granlocytopenia, hypersensitivity pncumonitis, keratitis, neplirotic syndrome, overlap syndrome, pigcon breeder's disease, pollinosis, idiopathic polyneuritis, urticaria, uveitis, juvenile dermatomyositisitis, and vitiligo.

In some embodiments, conditioned Treg cells disclosed herein are introduced into the subject to treat or modulate an autoimmune disorder or allergic disorder. For example, the subject may be afflicted with a disease characterized by having an ongoing or recurring autoimmune reaction or allergic reaction. In some embodiments, the modulating comprises inhibiting the autoimmune reaction or allergic reaction.

In some embodiments, conditioned Treg cells disclosed herein are administered to a subject for immunotherapy, such as, for example, in tumor surveillance, immunosuppression of cancers such as solid tumor cancers (e.g., lung cancer), and the suppression of in vivo alloresponses and autoimmune responses, including but not limited to, graft versus host disease (GVHD).

In some embodiments, the conditioned Treg cells disclosed herein may also be used to deliver suppressive or other biologic factors to sites of inflammation, such as but not limited to IL-4, stem cell growth factors, and angiogenesis regulators. For example, in some embodiments, the expanded, conditioned Treg cells can be transduced with genes encoding a desired biological factor, which the cell will then produce once within the subject, e.g., at the site of inflammation.

In some embodiments, the conditioned Treg cell compositions disclosed herein are indicated in infectious diseases in which the pathogenicity of the infections is not a result of the cytopathic effects of the pathogen but rather the tissue damage caused by the immunoinflammatory response to the infectious agent. In diseases, such as hepatitis B or C or HSV-induced corneal inflammation, therapy with the conditioned Treg cells disclosed herein provides a unique opportunity to control viral-induced immunoinflammatory disease. Viruses, such as Coxsackie, are known to cause pancreatitis and have been associated with the development of Type 1 Diabetes. Thus, Treg cell compositions as disclosed herein can be used to suppress local tissue damage caused by the infection and reduce the inflammation that incites autoimmune disorder development.

The subject methods find use in the treatment of a variety of different conditions and transplant situations in which the modulation of an aberrant immune response in a patient is desired. By way of example, but not by way of limitation, in the case of bone marrow or organ transplantation, composition comprising conditioned Treg cells disclosed herein may be administered during the time of surgery to prevent graft versus host disease in a transplant patient. To keep the cells at the site until completion of the surgical procedure, in some embodiments, it is convenient to administer the cells in a pharmaceutically acceptable carrier, such as an artificial gel, or in clotted plasma, or by utilizing other controlled release mechanism known in the art.

### III. EXAMPLES

The following examples illustrate select embodiments described herein. It is to be understood that the following examples are not limiting in any way, but may be adapted and applied as necessary and as understood by those of skill in the art.

### Example 1

### Generation of Conditioned Treg Cells Under Hypoxic Cell Culture Conditions

This example demonstrates the generation of conditioned Treg cells using hypoxic cell culture conditions.

Mouse natural Treg cells were isolated from the spleen and lymph nodes according to methods known in the art. Briefly, the methods were based on using monoclonal antibodies against cell surface proteins which are predominantly expressed on Treg cells. Using fluorochrome-conjugated antibodies, Treg cells were labeled and isolated by magnetic cell sorting. The cells were labeled with anti-HSA (CD24) and anti-CD8 mAbs using methods known in the art, and CD24+ and CD8− cells were depleted using magnetically assisted cell sorting (“MACS”) to enrich for CD4+ cells. CD25+ cells were further purified from this fraction by positive selection. The purity of CD4+CD25+ cells was >93%, with greater than 95% of cells CD4+CD25+FoxP3+.

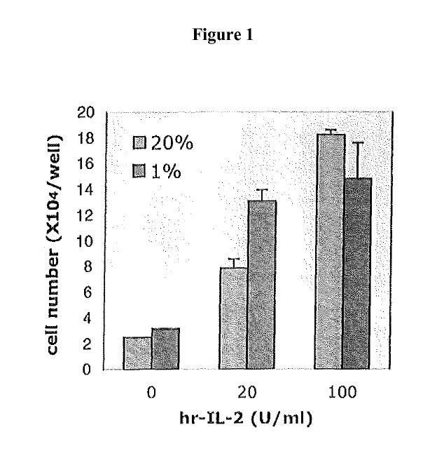

Purified CD4+CD25+ cells (1×10⁵ cells/well) were stimulated with immobilized anti-CD3 and anti-CD28 mAbs and cultured under 1% or 21% O₂ for 3 days. Culture media: RPMI1640 (Invitrogen) supplemented with 10% fetal calf serum (Hyclone); culturing condition: 37° C., 5% CO₂ using a NAPCO7000 incubator capable of controlling oxygen concentration; humidity: >95%, cell density: 5×105 cells/ml. Normoxic cell culture conditions were 5% C O₂, 95% air (21% oxygen). For hypoxic culture conditions, ambient air was diluted with pure nitrogen to reduce the O₂ concentration to 1%. Human recombinant IL-2 was added to the culture at 5-100 U/ml for the entire culture period. Treg cell proliferation was dependent on the dose of IL-2, but was unaffected by hypoxic culture conditions (FIG. 1).

These results show that the methods described herein are useful for the generation of conditioned Treg cells under hypoxic culture conditions. Such cells may be used for the treatment or prevention of diseases or conditions related to Treg cell levels, proliferation, or function.

### Example 2

### Up-Regulation of CTLA-4 Expression in Treg Cells Cultured Under Hypoxic

Conditions

Mouse natural Treg cells were isolated and conditioned as described above. Cells were cultured under 1% or 21% O₂, with 20 or 100 U/ml IL-2 for a period of 3 days. Cells were labeled with phycoerythrin-conjugated anti-CTLA-4 and allophycocyanin-conjugated anti-FoxP3 monoclonal antibodies using methods known in the art, and sorted using flow cytometric methods known in the art. A total of 50,000 events were acquired by FACSCalibur flow cytometer and the data was analyzed using CellQuest software.

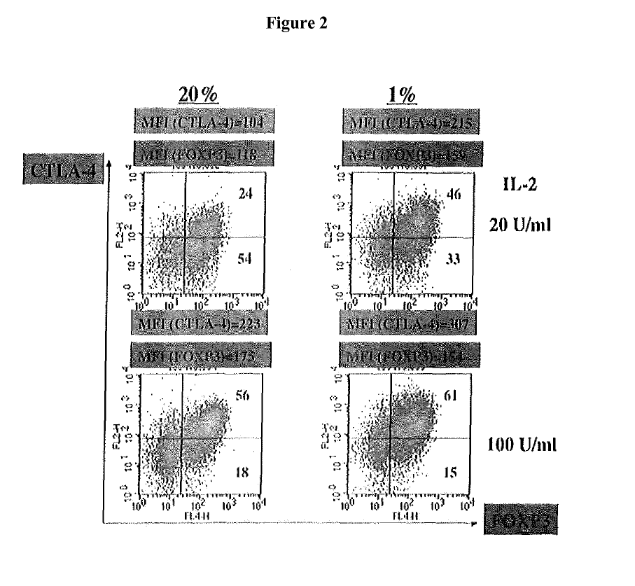

Hypoxic culture conditions induced a significant increase in Treg cell expression of CTLA-4 (CD 152) compared to normoxic controls (FIG. 2).

These results show that the methods disclosed herein are useful for generating conditioned Treg cells with elevated levels of CTLA-4 expression. Such cells may be used as agents for the prevention or treatment of diseases or conditions related to Treg cell levels, proliferation, function, or levels of Treg cell CTLA-4 expression.

### Example 3

### Up-Regulation of IL-10 Production by Treg Cells Cultured Under Hypoxic Conditions

Mouse natural Treg cells were isolated and conditioned as described above. Cells were cultured under 1% or 21% O₂, with 20 or 100 U/ml IL-2 for a period of 3 days. Levels of IL-10 in culture supernatants were determined using enzyme-linked immunosorbent assay (ELISA) methods known in the art.

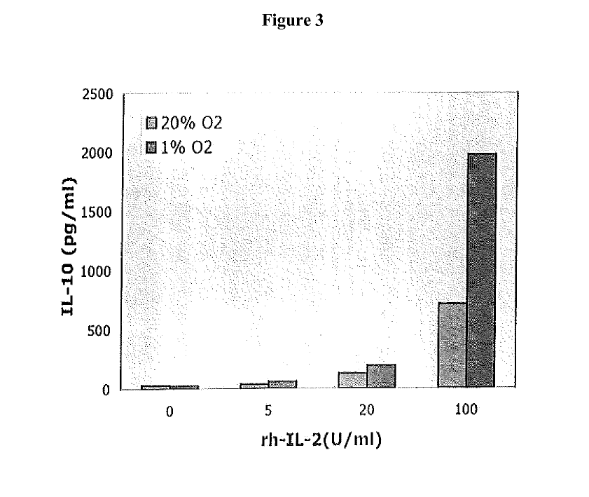

Hypoxic culture conditions induced a significant increase in Treg cell production of IL-10 compared to normoxic controls (FIG. 3).

These results show that the methods disclosed herein are useful for generating conditioned Treg cells with elevated levels of IL-10 production. Such cells may be used as agents for the prevention or treatment of diseases or conditions related to Treg cell levels, proliferation, function, or levels of Treg cell IL-10 production.

### Example 4

### Conditioned Treg Cells Possess Enhanced Immunoregulatory Activity

Purified Treg cells were stimulated and cultured as described above. Regulatory activity was determined by the inhibition of proliferative response of effector T cells. CD4⁺ CD25⁻ cells were purified from normal mouse and used as effector T cells.

After labeling with carboxyfluorescein succinimidyl ester (CFSE), the effector T cells were stimulated for 3 days with an immobilized anti-CD3 mAb in the presence of Treg cells cultured at either 1% or 21% O₂. After 3 days, CFSE levels in effector T cells were analyzed using FACSCalibur flow cytometer. A total of 50,000 events were acquired and the data was analyzed using CellQuest software.

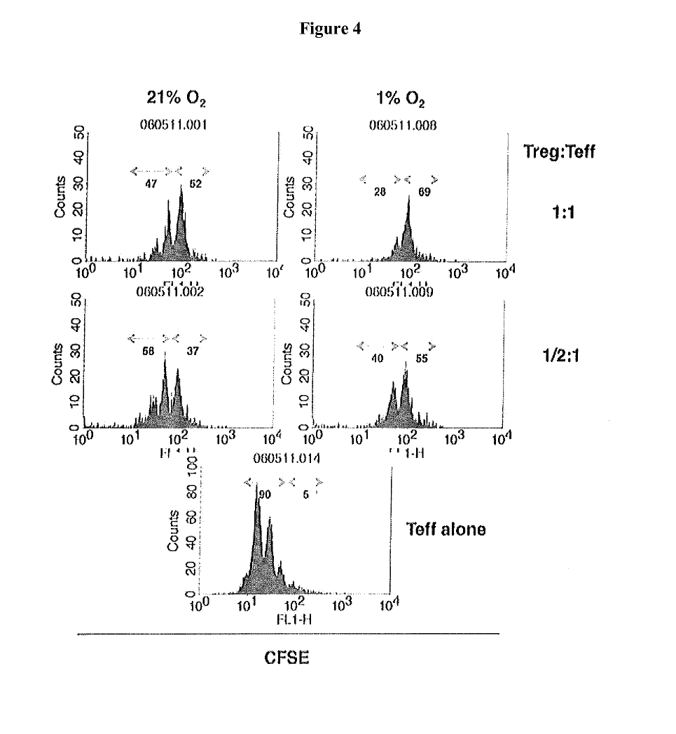

The stepwise dilution of CFSE fluorescence shown in FIG. 4 represents division of effector T-cells. The addition of Treg cells inhibited effector T-cell proliferation, with Treg cells cultured under hypoxic conditions showing a greater suppression of effector T-cell proliferation than Treg cells cultured under normoxic conditions (FIG. 4).

These results show that the methods disclosed herein are useful for generating conditioned Treg cells with enhanced immunoregulatory activity. Such cells may be used as agents for the prevention or treatment of diseases or conditions related to Treg cell immunoregulatory activity, such as relating to levels of T-cell proliferation.

### Example 5

### Generation of Conditioned Treg Cells Using Pharmacological Agents

This example will demonstrate the generation of conditioned Treg cells using pharmacological agents that up-regulate intracellular levels of cAMP.

Mouse natural Treg cells are isolated and cultured for a period of 3-7 days as described above in the presence of one or more pharmacological agents that increase cAMP levels. Such agents include, but are not limited to, G protein-coupled receptor ligands such as ligands of the A2A and A2B receptor (adenosine), ligands of the β-adrenergic receptor ligands (adrenaline), ligands of D1 and D5 receptors (dopamine), ligands of H2 receptor (histamine), ligands of DP, IP, EP2 and EP4 receptors (prostaglandins), ligands of 5-HT4, 5-HT6, 5-HT7 receptors (serotonin), ligands of PACT, VPAC1, VPAC2 and glucagon receptors (VIP, PACAP, glucagon). Additional exemplary agents include, without limitation phosphodiesterase inhibitors (including ibudilast)m cholera toxin, forskolin, caffeine, theophylline, bucladesine (dibutyryl cAMP, db cAMP), pertussis toxin, inhibitors of cyclic AMP dependent phosphodiesterase (PDE), and activators of Gs protein Inhibitors of cyclic AMP dependent phosphodiesterase (PDE) include but are not limited to PDE3 inhibitors (e.g., milrinone, inamrinone (formerly amrinone), cilostazol), PDE4 inhibitors (e.g. Ibudilast, roflumilast) and PDE5 inhibitors (e.g., sildenafil, tadalafil).

Inducers of cAMP are added at the beginning of the culture period of the Treg cells and are incubated for 4 days. Induction of cAMP may be confirmed by methods known in the art, for example, by brief incubation of purified CD4+CD25+ cells with the inducers of cAMP.

It is predicted that culturing Treg cells in the presence of one or more pharmacological agents that increase intracellular cAMP levels will have similar effects on Treg cells as culturing the cells under hypoxic conditions. It is predicted that the presence of the agent will not adversely impact cell proliferation, and will cause a significant up-regulation of CTLA-4 expression and IL-10 production.

These results show that the methods described herein are useful for the generation of conditioned Treg cells under culture conditions including one or more pharmacological agents that increase intracellular cAMP levels. Such cells may be used for the treatment or prevention of diseases or conditions related to Treg cell levels, proliferation, or function.

### Example 6

### Conditioned Treg Cells Suppress Auto-Immune Disease

This example will demonstrate the use of conditioned Treg cells for immunotherapy, such as, for example, in tumor surveillance, immunosuppression of cancers such as solid tumor cancers (e.g., lung cancer), and the suppression of in vivo alloresponses and autoimmune responses, including but not limited to, graft versus host disease (GVHD). The example is not to be construed as limited to conditions recited herein, but may be applied to any condition for which Treg cells confer a therapeutic or prophylactic benefit, as will be understood by one of skill in the art.

Conditioned Treg cells generated by culturing under hypoxic conditions or by the exposure to one or more pharmacological agents that increase intracellular cAMP levels are administered to a subject in need thereof in order to modulate the immune system, maintain or promote tolerance to self-antigens or foreign antigens, or to abrogate immune disorders. Subjects in need thereof include but are not limited to subjects having, suspected of having, or at risk of developing one or more immune-related diseases or conditions such as described herein.

Cells are administered to a subject in need thereof according to methods known in the art for the introduction of donor cells to a recipient host. The number of cells administered and the frequency of administration are determined according to guidelines known in the art including, but not limited to, characteristics of the recipient subject, prior pharmacological administrations to the subject, and the subject's response to the administration. By way of example, 4×10⁶ cells/kg are initially administered. Future or additional dosages may be increased or decreased, as determined e.g., by the administering physician. The patient is then monitored for abrogation of the immune disorder(s). Abrogation of the subject's immune disorder(s) is monitored using methods known in the art, including but not limited to measuring inflammatory response(s), the determining the number of immune cells present in the subject's circulation, and assessing immune disorder symptoms suffered by the subject.

What is claimed is:

1. A method for expanding T-regulatory cells, comprising:
culturing T-regulatory cells for at least 3 days under the following conditions:
0.5-5% oxygen,
5-100 U/ml of IL-2, and
in the presence of anti-CD3 and anti-CD28 antibodies.

2. The method of claim 1, wherein the T-regulatory cells to be expanded are CD4 positive and CD25 positive, and wherein at least 90% of the CD4 positive and CD25 positive T-regulatory cells are FoxP3 positive.

3. The method of claim 2, wherein at least 95% of the CD4 positive and CD25 positive T-regulatory cells are FoxP3 positive.

4. The method of claim 1, wherein the T-regulatory cells comprise human cells.

5. The method of claim 1, wherein the T-regulatory cells are cultured under 1% oxygen.

6. The method of claim 1, further comprising: contacting the T-regulatory cells with an agent that increases intracellular cyclic AMP (cAMP) levels.

7. The method of claim 1, comprising: after at least 3 days of culture, isolating T-regulatory cells expressing increased CTLA-4 and/or increased IL-10 levels as compared to control T-regulatory cells.

8. A method for expanding T-regulatory cells, comprising:
(a) culturing T-regulatory cells under normoxic conditions;
(b) culturing the T-regulatory cells of step (a) for at least 3 days under the following conditions:
0.5-5% oxygen,
5-100 U/ml of IL-2, and
in the presence of anti-CD2 and anti-CD28 antibodies.

9. The method of claim 8, wherein the T-regulatory cells to be expanded are CD4 positive and CD25 positive, and wherein at least 90% of the CD4 positive and CD25 positive cells are FoxP3 positive.

10. The method of claim 8, wherein the T-regulatory cells comprise human cells.

11. The method of claim 8, wherein the T-regulatory cells are cultured under 1% oxygen.

12. The method of claim 8, further comprising: contacting the T-regulatory cells with an agent that increases intracellular cyclic AMP (cAMP) levels.

13. A method for expanding T-regulatory cells, comprising:
culturing T-regulatory cells for at least 3 days in the presence of an agent that increases intracellular cyclic AMP (cAMP) levels.

14. The method of claim 13, wherein the agent that increases intracellular cAMP levels comprises one or more G protein-coupled receptor ligands.

15. The method of claim 14, wherein the G protein-coupled receptor ligand comprises one or more compounds selected from the group consisting of: ligands of the A2A and A2B receptor (adenosine), ligands of the β-adrenergic receptor ligands (adrenaline), ligands of D1 and D5 receptors (dopamine), ligands of H2 receptor (histamine), ligands of DP, IP, EP2 and EP4 receptors (prostaglandins), ligands of 5-HT4, 5-HT6, 5-HT7 receptors (serotonin), ligands of PACT, VPAC1, VPAC2 and ligands of glucagon receptors (VIP, PACAP, glucagon).

16. The method of claim 13, wherein the agent that increases intracellular cAMP levels comprises one or more compounds selected from the group consisting of phosphodiesterase inhibitors, ibudilast, cholera toxin, forskolin, caffeine, theophylline, bucladesine, dibutyryl cAMP, db cAMP, pertussis toxin, milrinone, inamrinone, sildenafil, tadalafil, and activators of Gs protein.

17. The method of claim 13, wherein the T-regulatory cells comprise human cells.

18. A method for modulating an autoimmune reaction in a subject in need thereof comprising:
administering the expanded T-regulatory cell of claim 1.

19. The method of claim 18, wherein the T-regulatory cell was obtained from the subject prior to expanding.

20. The method of claim 19, wherein the subject is suffering from an autoimmune disease selected from the group consisting of: Addison's disease, Celiac disease, dermatomyositis, Graves disease, Hashimoto's thyroiditis, multiple sclerosis, myasthenia gravis, pernicious anemia, reactive arthritis, rheumatoid arthritis, Sjogren syndrome, systemic lupus erythematosus, type I diabetes, graft versus host disease after organ transplant or bone marrow transplant.
